(19) United States
(12) Patent Application Publication
(10) Pub. No.: US 2012/0203239 A1
(43) Pub. Date: Aug. 9, 2012

(54) METHOD AND APPARATUS FOR CENTRATION OF AN OCULAR IMPLANT

(75) Inventors: John Allan Vukich, Madison, WI (US); Kyle Webb, Escondido, CA (US)

(73) Assignee: ACUFOCUS,INC., Irvine, CA (US)

(21) Appl. No.: 13/501,734

(22) PCT Filed: Oct. 13, 2010
(86) PCT No.: PCT/US10/52538

§ 371 Date: Apr. 12, 2012

(30) Foreign Application Priority Data
Oct. 13, 2009 (US) 61251253

(51) Int. Cl. A61F 9/00

(52) U.S. Cl. 606/107

(57) ABSTRACT

This application describes apparatuses and techniques for identifying and marking a position corresponding to the intersection of a patient's line of sight with the anterior surface of the human cornea to aid centration of an ocular implant. The apparatus can include a first optical subsystem configured to project a first light through an aperture and along an instrument axis such that the patient will observe an annulus around a disc when the patient's visual axis is substantially collinear with the instrument axis and the patient will not observe the annulus when the visual axis is not substantially collinear with the instrument axis. The apparatus may also include a second optical subsystem configured to project a second light along the instrument axis such that the second light appears to a clinician to be an annulus around the first light.

### CROSS-REFERENCE TO RELATED APPLICATIONS

This application claims the benefit of U.S. Provisional Application No. 61/251,253, filed Oct. 13, 2009, the entirety of which is hereby incorporated by reference.

### BACKGROUND OF THE INVENTION

1. Field of the Invention

This application relates to methods and apparatuses for aligning ocular implants (e.g., corneal inlays) with relevant anatomical structures (e.g., a patient's line of sight).

2. Description of the Related Art

Standard techniques for implanting corneal inlays involve the use of a surgical microscope. A Purkinje image is referenced through the microscope while moving the inlay into position. While this technique provides an approximation of alignment between the inlay and relevant ocular structures, various sources of error in alignment are introduced. For example, a parallax error can be introduced where a surgeon's vision is dependent on an image from a dominant eye.

### SUMMARY OF THE INVENTION

It has been discovered that the amount of error introduced in conventional techniques for corneal inlay placement may be too great for consistent performance of a small aperture device. In particular, standard techniques can introduce as much as 400 microns of error due to parallax alone. Therefore, there is a need for a more accurate method and apparatus for placement of a corneal inlay, such as an inlay that includes a small aperture. Such a method is not necessarily limited to inlays with small apertures, but can also be beneficial for any optical system that is designed to be positioned at a desired location relative to an anatomical landmark, such as the line of sight.

In certain embodiments, an apparatus for aligning a mask with a visual axis of an eye of a patient is provided. The apparatus can include a first optical subsystem configured to project a first light through an aperture and along an instrument axis such that the patient will observe an annulus around a disc when the patient's visual axis is substantially collinear with the instrument axis and the patient will not observe the annulus when the visual axis is not substantially collinear with the instrument axis. The diffraction aperture can have, for example, a diameter of less than about 300 microns, between about 100 microns and about 200 microns, and/or about 150 microns. The annulus around the disc may be an Airy pattern. The apparatus can also include a second optical subsystem configured to project a second light along the instrument axis such that the second light appears to a clinician to be an annulus around the first light. The apparatus may include a marker having a marker axis configured to be able to be aligned with the instrument axis such that the second light is at least partially projected onto the marker.

In certain embodiments, a method of aligning a visual axis of an eye with an instrument axis of an ophthalmic instrument is provided. The method may include projecting light through an aperture, passing the light along the instrument axis after projecting the light through the aperture, and causing movement of the eye to a position where the light that has passed through the aperture appears to the patient to include a disc surrounded by at least one ring. The diffraction aperture can have, for example, a diameter of less than about 300 microns, between about 100 microns and about 200 microns, and/or about 150 microns. The image of the disc surrounded by the at least one ring may be an Airy pattern. When the visual axis is not substantially collinear with instrument axis, the patient may not observe the at least one ring.

In certain embodiments, a method of increasing the depth of focus of an eye of a patient, the eye having a cornea and a visual axis is provided. The method may include aligning the visual axis with an instrument axis of an ophthalmic instrument. The aligning can include causing light to pass through an aperture and along the instrument axis, and causing movement of the eye to a position where the light that has passed through the aperture appears to the patient to include an image of a disc surrounded by at least one ring. After aligning the visual axis, the method may include projecting an annular image such that the annular image is visible to a clinician and is substantially aligned with the visual axis, and marking a surface of the cornea with a mark that corresponds to the annular image. The method can further include accessing a layer of the cornea, applying a mask comprising a pin-hole aperture having a mask axis to the eye of the patient, and aligning the mask with reference to the mark such that the mask axis is substantially collinear with the visual axis. The diffraction aperture can have, for example, a diameter of less than about 300 microns, between about 100 microns and about 200 microns, and/or about 150 microns. The method can further include aligning a marker having a marker axis such that the marker axis is substantially collinear with the instrument axis by projecting the annular image at least partially onto the marker. When the visual axis is not substantially collinear with instrument axis, the patient may not observe the at least one ring. The image of the disc surrounded by the at least one ring may be an Airy pattern.

### BRIEF DESCRIPTION OF THE DRAWINGS

FIG. 1A is a perspective schematic view of one embodiment of an ocular inlay centration apparatus taken generally from the position of a clinician in a procedure;

FIG. 1B is a perspective schematic view of the centration apparatus of FIG. 1 taken generally from the perspective of the patient;

FIG. 2A is an optical schematic of centration apparatus of FIGS. 1A and 1B.

FIG. 2B is a cross-section of A-A in FIG. 2A illustrating first and second centering cues.

FIG. 2C is a cross-section of B-B in FIG. 2A illustrating a marking implement.

FIG. 3 is a schematic of an optical system for generating a centering cue that will be visible to a patient during an implant procedure;

FIGS. 4A-C illustrate one embodiment of an optical system consistent with FIG. 3;

FIG. 4D illustrates one embodiment of a centering cue that can be visible to a patient during an implant procedure;

FIG. 5 is a schematic of an optical system for generating a centering cue that will be visible to a clinician;

FIGS. 6A-C illustrate one embodiment of an optical system consistent with FIG. 5;

FIG. 6D illustrates one embodiment of a centering cue that can be visible to a clinician in an implant procedure;

FIG. 7A and 7B illustrate the relationship of a plurality of centering cues that can be used in an ocular implant procedure;

FIG. 8A illustrates a position of a marking implement during one phase of a procedure;

FIGS. 8B-D illustrate various details of a marking implement that can be used with a centration apparatus; and

FIG. 9 illustrates a technique for centering the marking implement during an ocular implant procedure.

### DETAILED DESCRIPTION OF THE PREFERRED EMBODIMENT

This application describes an apparatus and technique for identifying and marking a position corresponding to the intersection of a patient's line of sight with the anterior surface of the human cornea. The line of sight is an imaginary line from the eye to a perceived object as referenced by the person doing the viewing. Additional apparatuses and techniques for aligning a patient's line of sight are described in U.S. Publication No. 2005/0046794, published on Mar. 3, 2005, the entirety of which is hereby incorporated by reference. Features of these additional apparatuses and techniques can be used in combination with and/or can be substituted for features described herein.

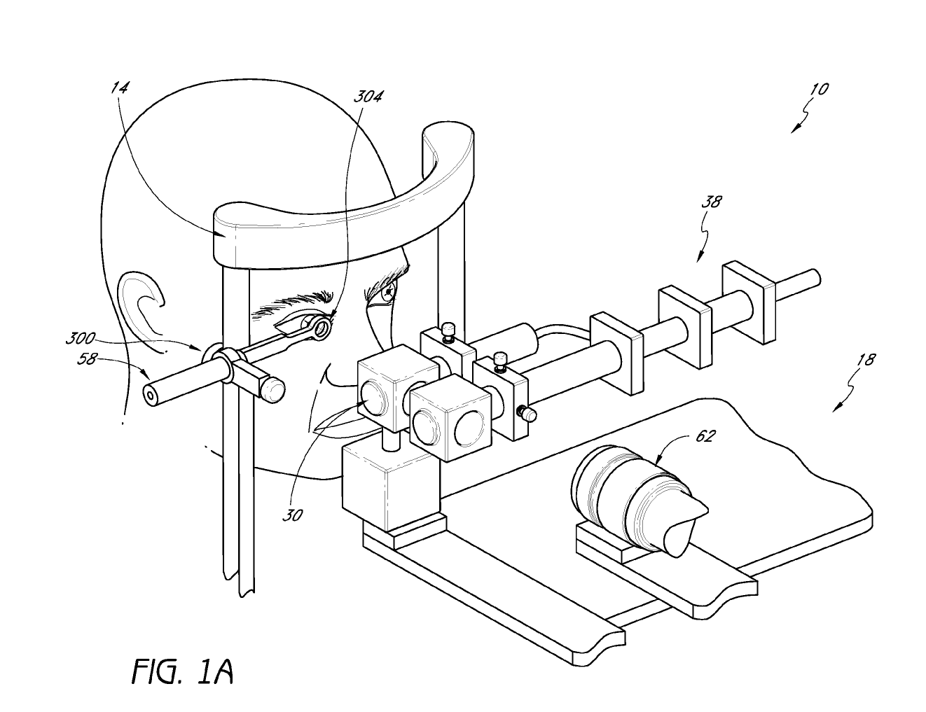

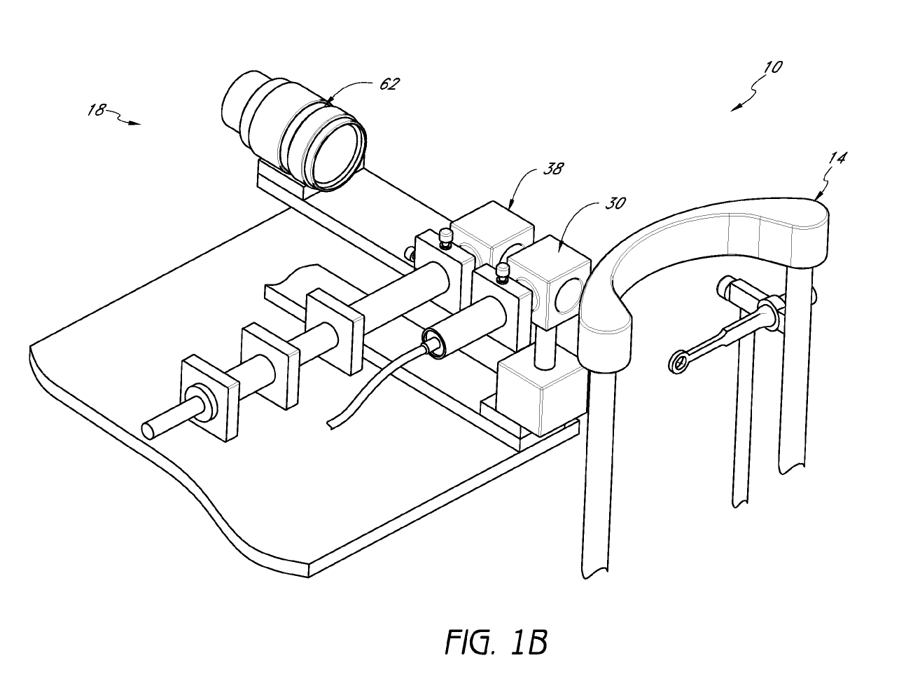

FIGS. 1A and 1B illustrate two views of an ocular inlay centration system 10. The system 10 includes a patient interface portion 14, a clinician data acquisition portion 18, and a plurality of optical components located therebetween. The optical components are configured to generate one or more centering cues that can be used during a procedure to align an ophthalmic device on the surface of or within the human cornea. In one embodiment, the system 10 also includes a first optical subsystem 38 for generating a first centering cue 42 that can be visible to a patient and a second optical subsystem 30 for generating a second centering cue 34 that can be visible to a clinician.

FIGS. 1A and 1B also show that in various embodiments the ocular inlay centration system 10 can include a marking system 58. The ocular inlay centration system 10 can optionally include a data acquisition system 62 that can be used to capture images of the patient's eye, the first centering cue 42, the second centering cue 34, and/or the marking system 58.

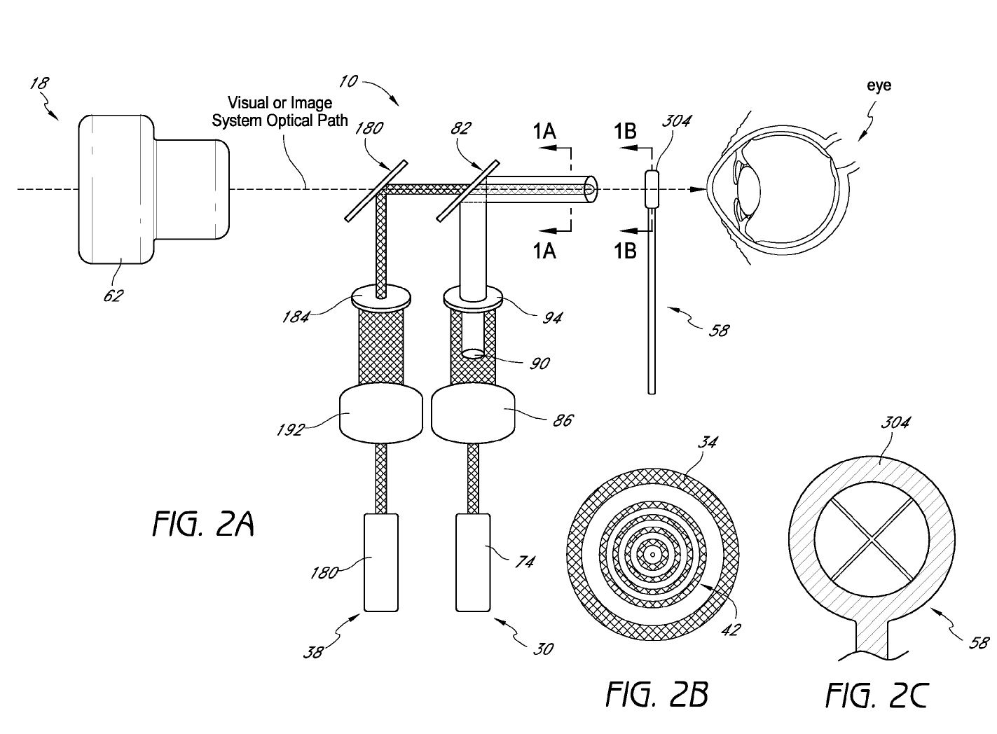

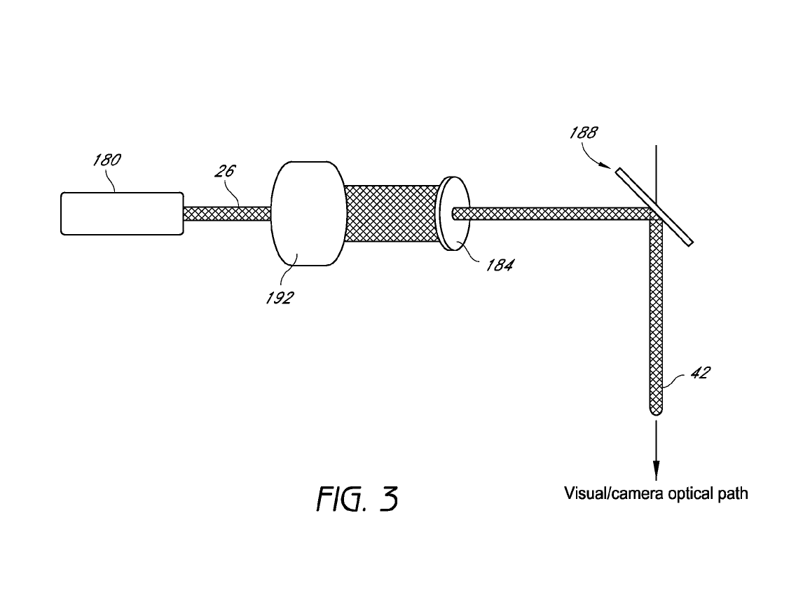

FIG. 2 is a schematic view of one embodiment of the system 10 that illustrates path of light 26 (represented by solid black) through the system 10. FIG. 3 shows schematically various details of one embodiment of the first optical subsystem 38. The subsystem 38 includes a light input 180, a light shaping subsystem 184, and a light directing device 188. The light directing device 188 can take any suitable form, but in one embodiment is a beam splitter, as discussed below.

In one embodiment, the light input 180 includes a green color (e.g. 535 nm wavelength) light input and a lens system 192 configured to expand the size, e.g. the width, a beam of light generated by the light.

The light shaping system 184 can include an opaque light blocking structure, such as an annular opaque mask configured to greatly reduce the width of the beam of light generated by the optical subsystem 38. More generally, the light shaping subsystem 184 preferably is configured to generate a visible light pattern that is configured to enhance the ability of the patient to center the patient's line of sight with respect to one or more components of the system 10. In one embodiment, an opaque annular mask with a very small diameter aperture can be placed in the path of the beam of the light generated by the light input 180. For example, a mask with a 150 μm diameter aperture can be used to produce a useful first centering cue 42. A mask with this diameter aperture can produce a series of concentric rings, such as is sometimes referred to as a Placido's disk, which can be visible to the patient as a bull's-eye pattern. More particularly, a mask can be configured to produce a series of concentric annular structures, e.g. concentric circular or ring-shaped areas. The configuration of the centering cue 42 is a function of several variables, including the distance that the light traverses from the light source 180 to the light shaping system 184, the distance that the light traverses from the light shaping system 184 to the patient, and the nature of the mask. For example, where a small aperture is formed in the mask through which light is to be directed, the size of the aperture can affect the centering cue 42. Centration using such a pattern is discussed in greater detail below.

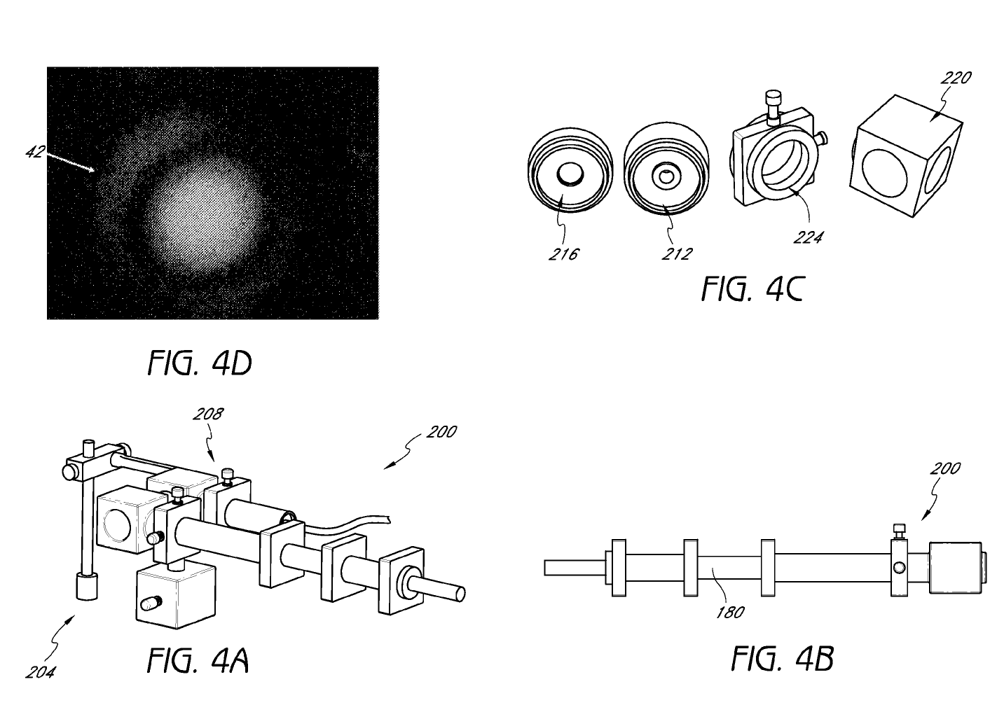

FIGS. 4A-C show further details of an optical subsystem 200, which is one implementation of the optical subsystem 38. The subsystem 200 can be used to generate the centering cue 42. The subsystem 200 includes a fixture 204 for supporting optical components 208. The fixture 204 can take any suitable form but preferably is arranged such that light directed through the components 208 is properly directed as discussed below. The optical components 208 will be described in the direction of travel of light therethrough. Light from the light input 180 enters a beam expander 212 in which the width, e.g. the diameter, of the light beam is increased.

Light exiting the beam expander 212 impinges upon a light shaping device 216. In this embodiment, the light shaping device 216 includes a small aperture having about a 150 μm diameter. The light shaping device 216 can be configured to produce any suitable visual pattern that can be concentrically or symmetrically disposed about the line of sight of the patient's eye. For example, the light shaping device 216 can be configured to produce a Placido's disk. Light exiting the light shaping device 216 is directed towards the patient's eye by a beam splitter 220. In some embodiments, one or more X-Y translation stages 224 are provided to adjust the position of one or more components, such as the aperture of the light shaping device 216.

FIG. 4D illustrates the centering cue 42 that is produced by the implementation of subsystem 200. As shown, the centering cue 42 includes a plurality of concentric arcuate structures that the patient can use to align the line of sight with an instrument. For example, in one technique, the centering cue 42 is configured to have a central disk-shaped area of light and one or more annular structures extending around the central area. In this technique, the patient achieves suitable centration of the patient's line of sight by moving their head and/or eye relative to the centering cue until the annular structure is concentrically disposed around the central area. A lack of centration exists when the annular structure is not concentric with the central disk-shaped area. In another technique, the first centering cue 42 is configured to have a central disk-shaped area of light. The patient achieves suitable centration of the patient's line of sight by moving their head and/or eye relative to the first centering cue 42 until one or more annular structures extending around the central area are observable by the patient. If the patient has not obtained centration of their line of sight, they will not be able to see the one or more annular structures. The annular structure can include one ring-shaped area in one variation that extends entirely around the central area. The annular structure can include two or more ring-shaped areas in other variations.

A lack of centration also may cause the central area to be altered from having a circular outer periphery to a more elongate or oblong outer periphery. In one variation, the centering cue is only a round disk and does not include any annular structures disposed therearound. In this variation, centration is achieved by the patient moving their head and/or eye until the disk of the centering cue is observed as having a round outer periphery. In the non-centered state, the central cue would be elongated in one direction, e.g., oblong.

The aperture can also have a variety of configurations that produce an Airy pattern. For example, the diameter of the aperture can be less than about 300 μm or less than about 200 μm. In another embodiment, the diameter of the aperture is between about 100 μm and about 200 μm. The first optical subsystem 38 can also include more than one aperture. For example, a plurality of apertures can be aligned along an axis which can further improve accuracy of the centration method.

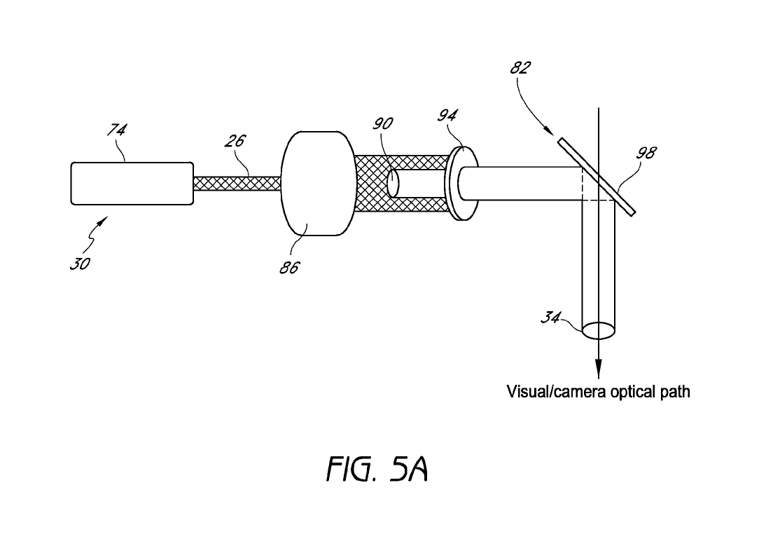

A second optical subsystem 30 can be used for generating a second centering cue 34 that can be visible to a clinician to assist the clinician in marking and/or implanting a corneal implant. FIG. 5 shows schematically various details of one embodiment of the second optical subsystem 30. The subsystem 30 includes a light input 74 a light shaping system 78 and a light directing device 82. The light input 74 can take any suitable form, such as providing a beam of light of sufficient width to create the centering cue 34. In one embodiment the light input 74 includes a red color (e.g. 635 nm wavelength) light input and the lens system 86 configured to expand the light input to width of greater than 3.8 mm. The width of the expanded beam can be within the range of 3.8 mm to about 4.5 mm. Preferably the light input 74 directs the light toward the light shaping system 78.

The light shaping system 78 can take any suitable form, but preferably is configured to produce the centering cue 34. For example in one embodiment the light shaping system includes a first light blocking member 90 and the second light blocking member 94. The first and second light blocking members 90, 94 preferably are configured to prevent transmission of a least a portion of the light from the light input 74. For example, the first light blocking member 90 can comprise an opaque disk having a diameter of about 3.8 mm. In one embodiment the opaque disk is a mask. The second light blocking member 94 can comprise an opaque structure having a central light transmitting aperture. In one embodiment the second light blocking number 94 includes a 4.5 mm diameter hole. In one embodiment, the light shaping system 78 is configured to produce an annulus of visible light having sufficient width to enable the clinician to see the relative position of the centering cues 34, 42. For example, the light shaping system 78 can be configured to produce an annulus of visible light having an inner diameter of about 3.8 mm and an outer diameter of about 4.5 mm.

The light directing device 82 can take any suitable form, for example incorporating a beam splitter 98 or any other structure configured to direct the centering cue 34 toward the patient's eye. One skilled in the art will appreciate that the light directing device 82 is optional.

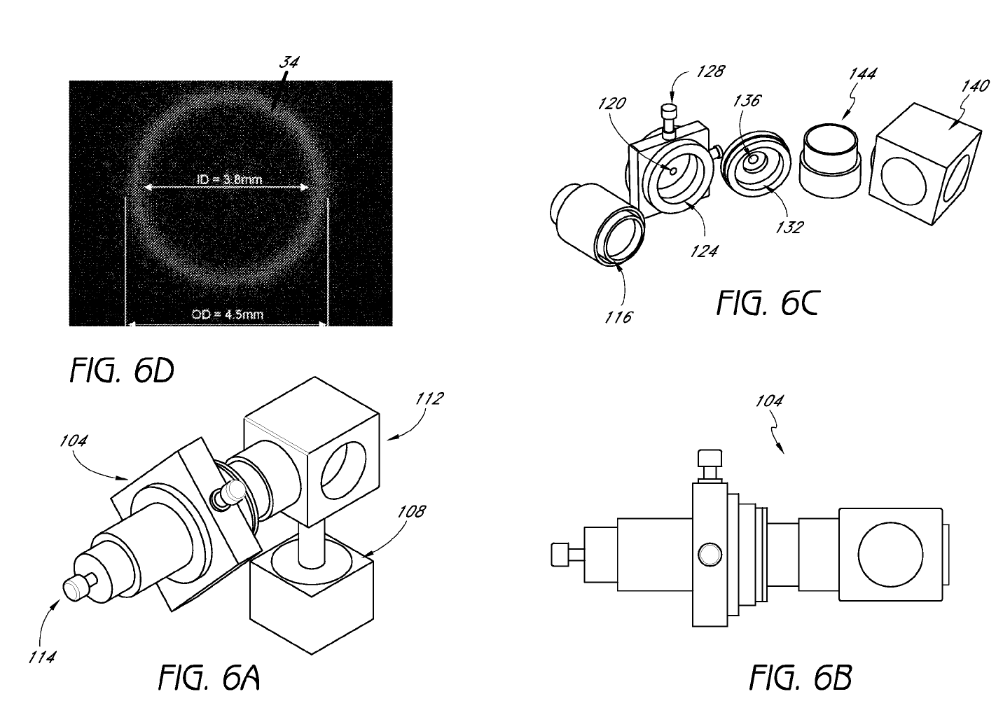

FIGS. 6A-C illustrate one implementation of an optical subsystem 104 consistent with the schematic diagram of FIG. 5. The subsystem 104 includes a fixture 108 and optical components 112 supported by the fixture 108. The optical components 112 will be described in the direction of travel of light through the optical system 104. Light from the light source 114 enters a beam expander 116 in which the width of the light is increased. After being increased, the light is directed from the beam expander 116 and impinges upon a first light shaping component that can include an opaque mask 120 that is supported by an X-Y translation stage 124. The X-Y translation stage 124 enables the position of the opaque mask 120 to be moved relative to the light. In various embodiments, the position of the mask 120 is predetermined and need not be adjustable using an X-Y translation stage. The X-Y translation stage 124 is configured to be moved in at least two directions by manipulation of the plurality of knobs 128. Other mechanisms for moving the stage 124 and the opaque mask 120 can implemented. For example, it may be advantageous to enable discrete precise movements in the X and/or Y directions. In one embodiment, a controlled movement is provided wherein a stepwise movement in either the X or Y direction can be achieved, with steps of about 1/20 mm. In another embodiment, discrete steps are provided such that 4-5 steps will cover modifications in position for a typical patient. In some patient populations, adjustment of the location of the centering cue 34 is expected to be less than about 0.2 mm. In some patient populations, adjustment of the location of the centering cue 34 is expected to be less than about 0.1 mm.

Light impinging on the opaque mask 120 will be blocked as light disposed peripherally around the mask 120 travels past the mask into a second light shaping component 132. The second light shaping component 132 includes a small aperture 136 that reduces the outer periphery of the outer peripheral sides of the light beam that exits the second light shaping component 132.

The light is then directed into a beam splitter 140 that is coupled with the second light shaping component 132 by an extension tube 144. The extension tube 144 can be eliminated but where present is useful for adjusting the relative position of the beam splitter 140 and the light source 114.

FIG. 6D illustrates one embodiment of the centering cue 34. As shown, the centering cue 34 can have a circular shape. As discussed above, the circular shape can be an annulus of any suitable dimension, such as for example having inner diameter of about 3.8 mm and outer diameter of about 4.5 mm.

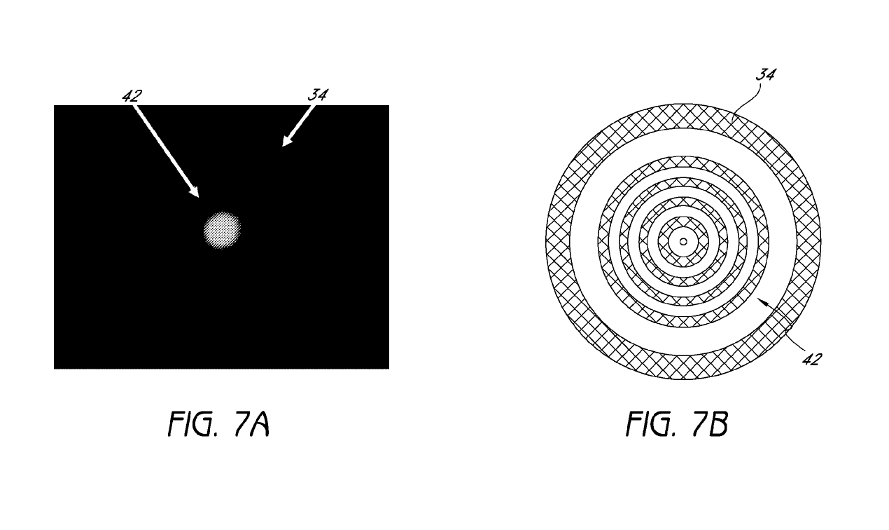

FIGS. 7A and 7B show the relationship of the second centering cue 34 and the first centering cue 42 according to the implementation of FIGS. 1-6. FIG. 7A is a photograph of the centering cues 34, 42, and FIG. 7B is an illustration. As shown, the second centering cue 34 is disposed concentrically with respect to the first centering cue 42.

One variation of the systems and methods disclosed herein will allow manual adjustment of the location of the centering cue 34. Such adjustment can be used to off-set the location of the centering cues 34, 42 such that they are not concentric, for example. In some patients, the location of the line-of-sight is not concentric with the pupil. The location of the line-of-sight can be off-set from the center of the pupil, e.g., slightly nasal and slightly inferior of the geometric center of the pupil. For such patients, it may be desirable to adjust the location of the centering cue 34 slightly such that the cornea can be marked at a location off-set from that illustrated by FIGS. 7A and 7B. For example, the X-Y translation stage 124 illustrated in FIGS. 6A-C could be used for manually adjusting the projected ring relative to the visual axis when the observed pupil is not coaxial and concentric to the visual axis. As discussed above, the adjustments can be small, discrete step-wise adjustments, such as of about 0.05 mm or less. Other amounts of movement of such steps could be provided. By providing discrete accurate off-set capability, the surgeon can be more accurate in positioning the centering cue 34. Also, data indicating the amount of offset for patients can be collected and analyzed to provide more information about the patient population.

In some methods, the determination of where to place an inlay is based upon both the location of the line-of-sight, as confirmed by the patient, and other characteristics, such as the position of a visible ocular feature, such as the pupil. The apparatus disclosed herein enables a surgeon to mark a cornea in connection with an inlay placement method taking into consideration both the patient confirmed line-of-sight and the location of a visible ocular feature, such as the inner edge of the iris.

Another advantage of the apparatus disclosed herein is that it enables a quick secondary confirmation of the location of the line-of-sight. For example, the patient can fixate on the centering cue 42 and the location of the line-of-sight indicated by this process can be recorded, for example by the data acquisition system 62. Thereafter, a patient can look away from the cue 42. After looking away from the cue, the patient can fixate on the centering cue 42 again. The location of the line-of-sight indicated by the second fixation can be recorded, for example by the data acquisition system 62. The first and second location of the line-of-sight can be used to determine an appropriate location to mark on the cornea, alone or in combination with the position of one or more visible ocular features.

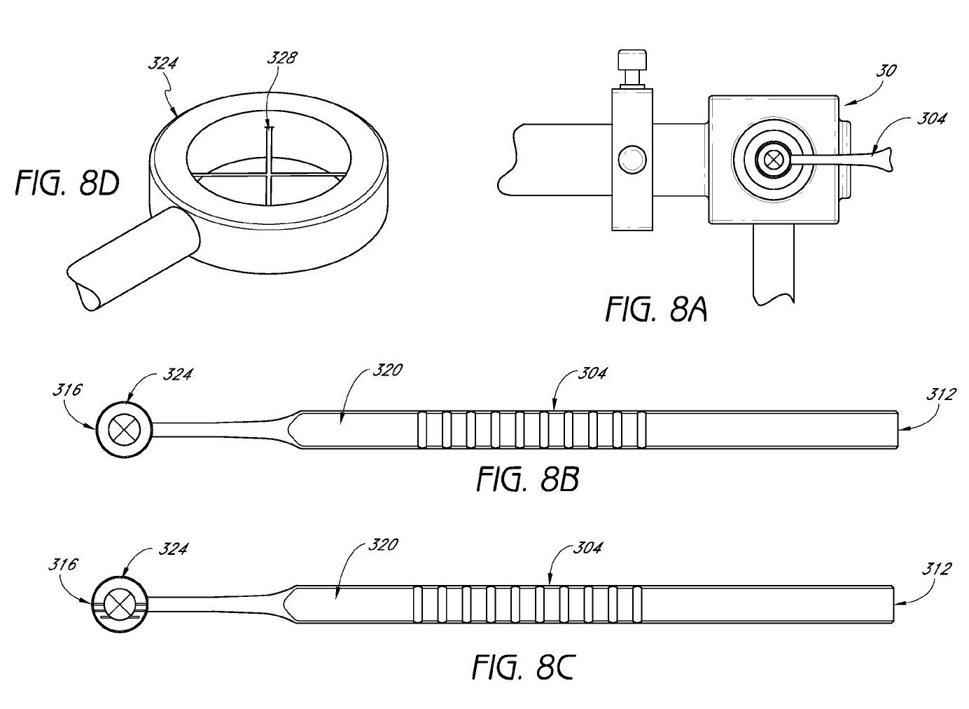

FIGS. 8A-D illustrate one embodiment of the marking system 58. As shown in FIG. 1, marking system 58 includes a fixture 300 that can be configured to position a marking implement 304 relative to the patient's eye. The fixture 300 can take any suitable form such as for example immobilizing or substantially fixing the position of the marking implement 304. In some embodiments, the fixture 300 is configured to permit movement of the marking implement 304 in at least one degree of freedom, e.g. along an axis corresponding to or coaxial with the line of sight of the eye. In the embodiment, the fixture 300 enables the marking implement 304 to be translated from a position spaced away from an outside surface of the cornea into engagement with the outside surface of the cornea.

The marking implement 304 includes a proximal end 312, a distal end 316, and elongated body 320 extending therebetween. The marking implement 304 includes a marking ring 324 disposed adjacent the distal end 316. The marking ring 324 can have an annular shape and can include a crosshair structure 328 disposed therein. The crosshair structure 328 is optional in some embodiments. In one embodiment, the inner diameter of the annular shaped marking ring 324 has a diameter that corresponds to an inner portion of the marking cue 34. For example, the marking ring can have an inner diameter of about 3.8 mm such that all or substantially all of the marking cue 34 shines on an inner peripheral portion of the marking ring 324 in use. The marking ring 324 can have ink disposed on the ring such that when the marking ring 324 is placed in contact with the eye, at least some ink will be left on the eye to provide the clinician a mark to provide assistance with aligning a mask or corneal inlay. The mask or corneal inlay can include those described in U.S. Publication No. 2006/0265058 and International Application No. PCT/US2010/045541 filed Aug. 13, 2010, the entirety of each of which is hereby incorporated by reference.

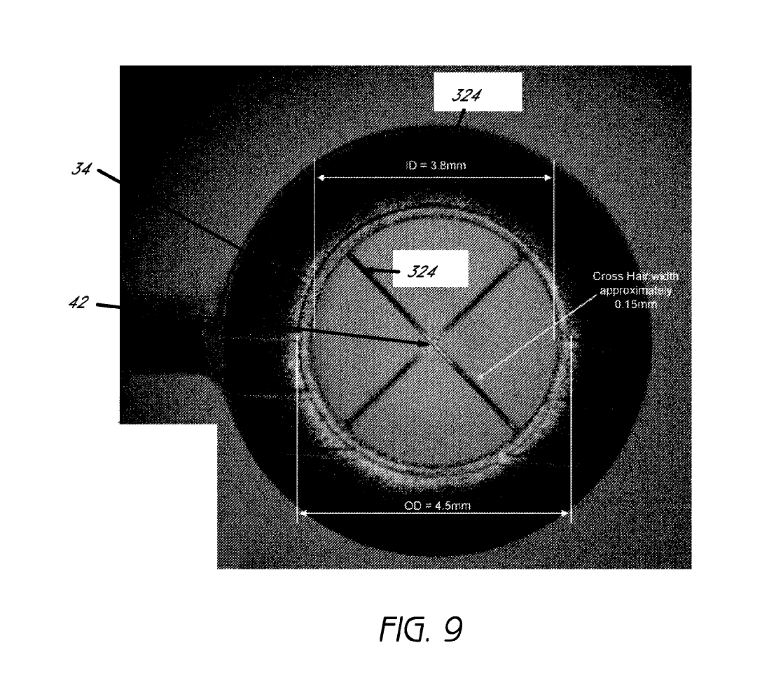

FIG. 9 shows the relative position of the marking cues 34, 42 with respect to the marking implement 304 during use of the system 10. As shown, the marking cue 42 is generally centered on the center of a crosshair 328 and the marking cue 34 is centrally disposed around the inner periphery of the marking ring 324. In some embodiments and techniques, the sufficient confirmation of positioning of the marking ring 324 can be achieved by confirming the position of the cue 34 relative to the ring 324, thus the crosshair structure 328 is optional.

The above-described apparatuses can be used for identifying and marking the line of sight, e.g., for centration of a device on the anterior surface of or within the human cornea.

A far field pinhole wavefront diffraction pattern image (e.g., a classic Placido's disk image) is created and presented to the patient in one embodiment. The patient centers on the image, as discussed above, and visual fixation of the presented target is achieved. The patient's line of sight is established upon visual fixation. In particular, the patient moves their head and/or eye until a centering cue has a predetermined shape or configuration. At this moment, visual fixation of the line of sight has occurred. When the centering cue has the predetermined shape or configuration, the patient's line of sight is at a known location relative to other components of the centration system. For example, where the centering cue is viewed as a plurality of concentric arcuate structures (e.g., circular peripheries) the line of sight of the patient's eye can be confirmed to be centered or fixated. In another example, the direction that the patient is looking is altered until a disk-shaped centering cue changes from being elongate in at least one direction to being observed to have a substantially circular outer periphery. Thus the apparatus and method provide a self centering target image.

A ring of light with a selected diameter and line thickness is optically imaged to be concentric around the far field wavefront image target or other target. Green light wavelength is selected to differ from wavefront target image, which can be red, so that visual discrimination can be apparent between the two separately optically produced images. Other techniques can be used to prevent a patient from confusing the light projected as the two centering cues. For example, the same color light or light with overlapping wavelengths can be used in one variation where the two centering cues are not illuminated at the same time. The second centering cue (e.g., ring of light) may also be substantially outside the patient's field of view so that the patient only observes the first centering cue (e.g., wavefront diffraction pattern image).

A custom corneal marker with a flat reflective surface is placed in the path of the ring of light. The corneal marker marks a location for the patient that corresponds to the line of sight target image. The flat reflective surface reflects the ring light. The surgeon visually adjusts the corneal marker location to obtain concentric alignment between the ring light projected image and the target image. The surgeon marks the cornea with alignment guidance of the ring light.

In one technique, the patient sits and positions his or her head in a chin rest platform. The patient obtains visual observation of a Placido's disk and fixates on the target. Upon patient acknowledgment of the target fixation, the surgeon places custom corneal marker in positions near the cornea and obtains reference ring light alignment. Alignment is obtained when corneal marker is aligned concentric with the ring light. The surgeon then marks the corneal while maintaining concentric alignment of the ring light and the corneal marker.

Although the foregoing description is of a slit-lamp type arrangement, in another embodiment, the patient is positioned prone on an operating table. Similar components would be incorporated into such a system, as will be appreciated by one skilled in the art. In this technique, the patient lies flat on his or her back directing their attention to a projected image of the centering cue 42 or other fixation target. A surgical microscope can be used to confirm alignment of one, two ore more than two centering cues while the patient lies on their back, looking upward. Thereafter, the cornea is marked to provide an indication useful in a later portion of a procedure of where to position an inlay.

1. An apparatus for aligning a mask with a visual axis of an eye of a patient, the apparatus comprising:
a first optical subsystem configured to project a first light through a diffraction aperture and along an instrument axis such that the patient will observe an annulus around a disc when the patient's visual axis is substantially collinear with the instrument axis and the patient will not observe the annulus when the visual axis is not substantially collinear with the instrument axis.

2. The apparatus of claim 1, wherein the diffraction aperture comprises a diameter of less than about 300 microns.

3. The apparatus of claim 1, wherein the diffraction aperture comprises a diameter of between about 100 microns and about 200 microns.

4. The apparatus of claim 1, wherein the diffraction aperture comprises a diameter of about 150 microns.

5. The apparatus of claim 1, further comprising a second optical subsystem configured to project a second light along the instrument axis such that the second light appears to a clinician to be an annulus around the first light.

6. The apparatus of claim 5, further comprising a marker having a marker axis configured to be able to be aligned with the instrument axis such that the second light is at least partially projected onto the marker.

7. The apparatus of claim 1, wherein the annulus around the disc comprises an Airy pattern.

8. A method of aligning a visual axis of an eye with an instrument axis of an ophthalmic instrument, the method comprising:
projecting light through a diffraction aperture;
passing the light along the instrument axis after projecting the light through the aperture; and
causing movement of the eye to a position where the light that has passed through the aperture appears to the patient to include a disc surrounded by at least one ring.

9. The method of claim 8, wherein the diffraction aperture comprises a diameter of less than about 300 microns.

10. The method of claim 8, wherein the diffraction aperture comprises a diameter of between about 100 microns and about 200 microns.

11. The method of claim 8, wherein the diffraction aperture comprises a diameter of about 150 microns.

12. The method of claim 8, wherein when the visual axis is not substantially collinear with instrument axis, the patient does not observe the at least one ring.

13. The method of claim 8, wherein the image of the disc surrounded by the at least one ring comprises an Airy pattern.

14. A method of increasing the depth of focus of an eye of a patient, the eye having a cornea and a visual axis, the method comprising:
aligning the visual axis with an instrument axis of an ophthalmic instrument, said aligning comprising:
causing light to pass through a diffraction aperture and along the instrument axis; and
causing movement of the eye to a position where the light that has passed through the aperture appears to the patient to include an image of a disc surrounded by at least one ring;
after the aligning the visual axis, projecting an annular image such that the annular image is visible to a clinician and is substantially aligned with the visual axis;
marking a surface of the cornea with a mark that corresponds to the annular image;
accessing a layer of the cornea;
applying a mask comprising a pin-hole aperture having a mask axis to the eye of the patient; and
aligning the mask with reference to the mark such that the mask axis is substantially collinear with the visual axis.

15. The method of claim 14, wherein the diffraction aperture comprises a diameter of less than about 300 microns.

16. The method of claim 14, wherein the diffraction aperture comprises a diameter of between about 100 microns and about 200 microns.

17. The method of claim 14, wherein the diffraction aperture comprises a diameter of about 150 microns.

18. The method of claim 14, further comprising aligning a marker having a marker axis such that the marker axis is substantially collinear with the instrument axis by projecting the annular image at least partially onto the marker.

19. The method of claim 14, wherein when the visual axis is not substantially collinear with instrument axis, the patient does not observe the at least one ring.

20. The method of claim 14, wherein the image of the disc surrounded by the at least one ring comprises an Airy pattern.
